(19) United States
(12) Patent Application Publication
(10) Pub. No.: US 2026/0363910 A1
(43) Pub. Date: Nov. 19, 2026

(54) CONJUNCTIVOCYSTORHINOSTOMY TUBE RESISTANT TO INFECTION AND BIOFILM

(71) Applicant: The Regents of the University of California, Oakland, CA (US)

(72) Inventors: Jeremiah Tao, Irvine, CA (US); Albert F. Yee, Irvine, CA (US); Teresa Chen, Irvine, CA (US)

(21) Appl. No.: 19/679,362

(22) Filed: May 15, 2026

Related U.S. Application Data
(60) Provisional application No. 63/806,269, filed on May 15, 2025.

(51) Int. Cl. A61F 9/007; A61L 27/54

(52) U.S. Cl. CPC A61F 9/00772; A61L 27/54

(57) ABSTRACT

An antimicrobial structure for insertion into a conjunctivodacryocystorhinostomy tube. The structure includes a first layer comprising a solid material. The structure further includes a second layer disposed on the first layer, having an antimicrobial material or another thermoplastic or thermoset material, and a plurality of nanopillars disposed on a surface of the antimicrobial material.

### CROSS-REFERENCES TO RELATED APPLICATIONS

This application is a non-provisional and claims benefit of U.S. Provisional Application No. 63/806,269 filed May 15, 2025, the specification of which is incorporated herein in its entirety by reference.

### FIELD OF THE INVENTION

The present invention is directed to a medical device for tearing that prevents biofilm build-up.

### BACKGROUND OF THE INVENTION

A conjunctivocystorhinostomy (cDCR) is the surgical connection between the conjunctiva to the lacrimal sac to the nose for patients with tearing due to an obstruction in the tear drainage system, specifically in the canaliculus. Previous cDCR tubes are made of borosilicate glass. Nanopillars are nanostructures that are pillar- or conical-shaped and about 100 to 500 nanometers in diameter. They are arranged in an approximately closest pack order and can inhibit the growth of bacteria and fungi upon contact, which prevents biofilm formation. Current solutions for biofilm build-up include removing the borosilicate glass tubes in the clinic or in the operating rooms and cleaning or replacing the tubes. Removing and replacing the glass tubes can be uncomfortable, painful, and costly. Thus, there exists a present need for a conjunctivodacryocystorhinostomy tube configured to minimize the buildup of biofilm over time.

### BRIEF SUMMARY OF THE INVENTION

It is an objective of the present invention to provide devices, systems, and methods that allow for a conjunctivodacryocystorhinostomy tube configured to minimize the buildup of biofilm over time, as specified in the independent claims. Embodiments of the invention are given in the dependent claims. Embodiments of the present invention can be freely combined with each other if they are not mutually exclusive.

The present invention features an antimicrobial structure for insertion into a conjunctivodacryocystorhinostomy tube. The structure may comprise a first layer comprising a solid material having a plurality of nanopillar protrusions. The structure may further comprise a second layer disposed on the first layer, comprising an antimicrobial chitosan material disposed on the plurality of nanopillars. The second layer may also comprise a plurality of polar polymers disposed on the nanopillars. Chitosan is a coating atop the nanopillars formed on the first layer.

The unique and novel features of this cDCR tube are its polar polymeric material with nanopillars. The nanopillars are rendered highly polar by coating them with chitosan. The present invention implements Nanoimprint Lithography to place nanopillars on the surfaces of thin films of polymers with polar chemical groups as side groups or along the main chain of the molecule, including, but not limited to chitosan, thermoplastic polyurethane (TPU), bisphenol-A polycarbonate (PC), poly(ethylene terephthalate) (PET), and Poly(ethylene terephthalate-co-glycol) (PETG). The imprinted film is then rolled up and inserted into a glass or polymeric tube. The polymer used as a supporting substrate may comprise TPU, PC, PET, or PETG. The first layer may comprise any combination of these polymers, which may be imprinted with nanopillars. Then, a second layer comprising chitosan, may be coated onto the first layer.

One of the unique and inventive technical features of the present invention is the implementation of a nanopillar layer disposed on the interior of a conjunctivodacryocystorhinostomy tube for tearing treatment. Without wishing to limit the invention to any theory or mechanism, it is believed that the technical feature of the present invention advantageously provides for the minimization of bacterial and fungal growth and biofilm buildup in a conjunctivocystorhinostomy treatment tube, reducing the need for said tube to be periodically removed and cleaned, or replaced. None of the presently known prior references or works has the unique inventive technical feature of the present invention.

Furthermore, the inventive technical features of the present invention contributed to a surprising result. One of ordinary skill in the art would expect that antimicrobial nanopillar films, such as chitosan, could not be used for forming a film over nanopillars because the material is known to be soft and difficult to work with. These films are well-known to furl up and adhere to themselves, rendering the interior nanopillar surface useless. The present invention implements a bilayer “sandwich” of a chitosan film and a polar thermoplastic layer upon which the chitosan layer is applied. Surprisingly, this allows for the present invention to achieve an antimicrobial structure able to be disposed within a conjunctivodacryocystorhinostomy tube for minimizing biofilm buildup. Thus, the inventive technical feature of the present invention contributed to a surprising result.

Furthermore, antimicrobial polymer films require crosslinking to adhere to a solid substrate capable of allowing the film to remain stable in the conjunctivodacryocystorhinostomy tube. In many cases, one or ordinary skill in the art would expect that crosslinking the antimicrobial film would render it inert in terms of antimicrobial capabilities. Surprisingly, the present invention is able to achieve this adhesion and a solid structure for conjunctivodacryocystorhinostomy procedures with antimicrobial capabilities. Oxygen plasma treatment may be used to adhere chitosan to the thermoplastic nanopillar surface.

Any feature or combination of features described herein is included within the scope of the present invention, provided that the features included in any such combination are not mutually inconsistent, as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings, in which:

FIG. 1 shows a schematic diagram of a process for forming the antimicrobial conjunctivodacryocystorhinostomy tube structure comprising an antimicrobial layer disposed atop nanopillars.

FIG. 2A shows a schematic diagram of the antimicrobial structure having an antimicrobial layer disposed on nanopillars formed on the molding layer.

FIG. 2B shows a scan of the mold used to form the nanopillars with the antimicrobial coating in the pores of the mold.

FIG. 2C shows a schematic diagram of the antimicrobial coating disposed in the inverted nanopillars of the molding layer of the present invention.

FIG. 3A shows a schematic diagram of the antimicrobial structure of the present invention. The structure comprises a substrate layer having a plurality of nanopillar protrusions, and a thin layer of chitosan disposed atop the nanopillars.

FIG. 3B shows a schematic diagram of a system comprising a conjunctivodacryocystorhinostomy tube and an antimicrobial structure of the present invention. The structure comprises a substrate layer having a plurality of nanopillar protrusions, and a thin layer of chitosan disposed atop the nanopillars.

FIG. 3C shows a flowchart of a method for fabricating the antimicrobial structure comprising an antimicrobial layer disposed atop nanopillars of the present invention.

FIG. 4A shows a first embodiment of the present invention, where the antimicrobial structure comprising an antimicrobial layer disposed atop nanopillars is inserted into a pre-set conjunctivodacryocystorhinostomy tube.

FIG. 4B shows a second embodiment of the present invention, where the antimicrobial structure comprising an antimicrobial layer disposed atop nanopillars acts as a self-standing conjunctivodacryocystorhinostomy tube.

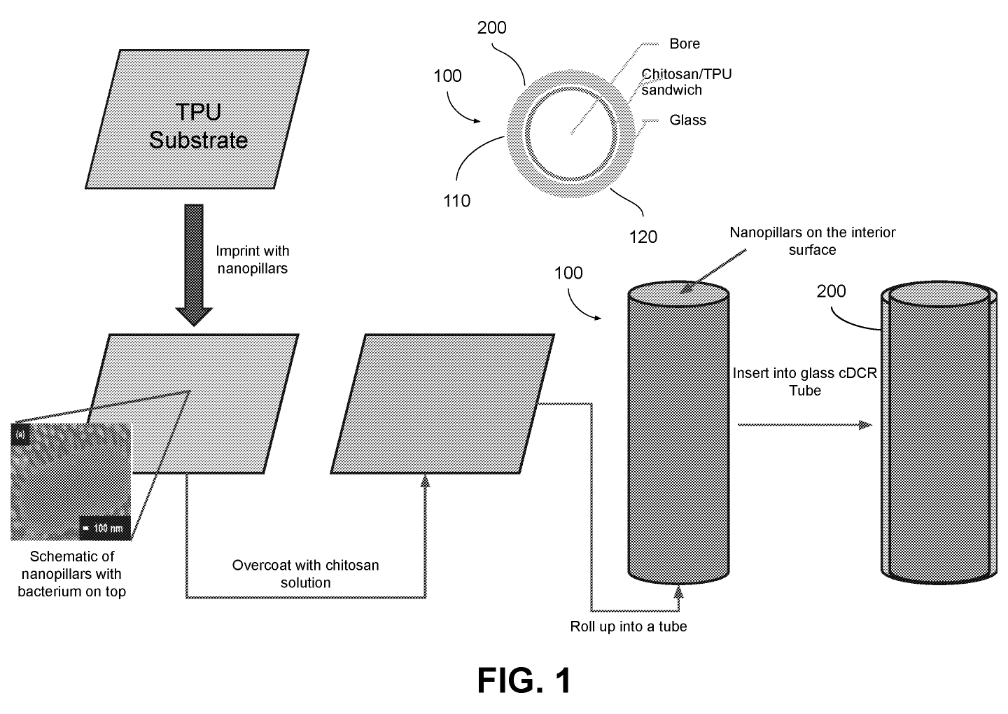

### DETAILED DESCRIPTION OF THE INVENTION

Following is a list of elements corresponding to a particular element referred to herein:
100 structure
110 substrate layer
112 interior of substrate layer
114 exterior of substrate layer
120 antimicrobial layer
125 nanopillars
130 molding layer
200 conjunctivodacryocystorhinostomy tube
202 interior of conjunctivodacryocystorhinostomy tube
1000 system
2000 patient

The term “nanopillar” is defined herein as cylindrical pillar- or cone-shaped nanostructures approximately 100 to 900 nanometers in diameter that can be grouped together in regular or irregular approximate arrays.

The term “conjunctivodacryocystorhinostomy tube” is defined herein as a thin glass or polymeric tube used to create a new channel for draining tears in a patient.

In some embodiments, the present invention features an antimicrobial structure (100) configured to create a channel in soft tissue from an eyelid to a nasal passage of a patient (2000) for tear liquid drainage, the antimicrobial structure (100) including: a) a substrate layer (110) including a solid material arranged in a hollow cylindrical shape having an interior surface (112) and an exterior surface (114), and a plurality of nanopillars (125); and b) an antimicrobial layer (120) disposed on the interior surface of the substrate layer (110), including an antimicrobial film disposed on the plurality of nanopillars (125).

In some embodiments, the present invention features an antimicrobial structure (100), wherein the solid material includes thermoplastic polyurethane, polycarbonate, or a combination thereof.

In some embodiments, the present invention features an antimicrobial structure (100), wherein the antimicrobial structure (100) is configured to be inserted into a conjunctivodacryocystorhinostomy tube (200) having an interior surface (202) such that the exterior surface (114) of the substrate layer (110) adheres to the interior surface (202) of the conjunctivodacryocystorhinostomy tube (200).

In some embodiments, the present invention features an antimicrobial structure (100), wherein the antimicrobial structure (100) is configured to be inserted directly into the soft tissue and lacrimal bone of the patient (2000) such that the exterior surface (114) of the substrate layer (110) contacts the soft and hard tissue of the patient (2000).

In some embodiments, the present invention features an antimicrobial structure (100), wherein the plurality of nanopillars (125) are formed on the solid layer (110) from a molding layer (130) including a plurality of pores including V-shaped pores, or cylindrical-shaped pores, such that the plurality of nanopillars (125) are formed within the plurality of pores.

In some embodiments, the present invention features an antimicrobial structure (100), wherein the molding layer (130) includes anodized aluminum oxide (AAO) or anodized titanium oxide (ATO). In some embodiments, the present invention features an antimicrobial system (1000) configured to create a channel in soft tissue from an eyelid to a nasal passage of a patient (2000) for tear liquid drainage, the antimicrobial system (1000) including: a) a conjunctivodacryocystorhinostomy tube (200); and b) an antimicrobial structure (100) rolled into a hollow cylindrical shape and disposed within the conjunctivodacryocystorhinostomy tube (200), the antimicrobial structure (100) including: i) a substrate layer (110) including a solid material arranged in a hollow cylindrical shape having an interior surface (112) and an exterior surface (114), and a plurality of nanopillars (125); and ii) an antimicrobial layer (120) disposed on the interior surface of the substrate layer (110), including an antimicrobial film disposed on the plurality of nanopillars (125).

In some embodiments, the present invention features an antimicrobial system (1000), wherein the solid material includes thermoplastic polyurethane, polycarbonate, polyethylene terephthalate, or polyethylene terephthalate-co-glycolate, or other polar thermoplastics such as polyamides, polyarylamides, polyketones, polyarylesters, or copolymers thereof. In some embodiments, the present invention features an antimicrobial system (1000), wherein the conjunctivodacryocystorhinostomy tube (200) includes borosilicate glass, steel, aluminum, plastic, or a combination thereof.

In some embodiments, the present invention features an antimicrobial system (1000), wherein the plurality of nanopillars (125) are formed on the solid layer (110) from a molding layer (130) including a plurality of pores including V-shaped pores, cylindrical-shaped pores, such that the plurality of nanopillars (125) are formed within the plurality of pores.

In some embodiments, the present invention features an antimicrobial system (1000), wherein the molding layer (130) includes AAO or ATO.

In some embodiments, the present invention features a method for fabricating an antimicrobial structure (100) for insertion into a conjunctivodacryocystorhinostomy tube (200), the method including: a) providing a molding layer (130) including a plurality of pores; b) casting a first solution or melt onto the molding layer (130); c) curing the first solution or melt such that a solid layer (110) is formed, the solid layer (110) including a plurality of nanopillars (125) formed in the plurality of pores; d) lifting the solid layer (110) from the molding layer (130) to result in the structure with a plurality of nanopillars on the surface (100); and e) applying a second solution including chitosan to the nanopillar layer (110), resulting in the antimicrobial structure (100).

In some embodiments, the present invention features a method, wherein the molding layer (130) includes AAO or ATO.

In some embodiments, the present invention features a method, wherein the first solution or melt includes an emulsion of lecithin or 1H,1H,2H,2H-Perfluorodecyltriethoxysilane (PFDTES).

In some embodiments, the present invention features a method, wherein the second solution further includes citric acid, acetic acid, or a combination thereof.

In some embodiments, the present invention features a method that further includes applying pressure via flat metal plates parallel to the AAO or ATO mold for creating the antimicrobial structure (100).

In some embodiments, the present invention features a method further including applying a dilute NaOH solution to the chitosan layer (120), followed by applying oxygen plasma treatment to the chitosan layer (120).

In some embodiments, the present invention features a method further including rolling the antimicrobial structure (100) into a hollow cylindrical shape such that the plurality of nanopillars (125) face inwards in the hollow cylindrical shape, and inserting the cylinder into the conjunctivodacryocystorhinostomy tube (200).

In some embodiments, the present invention features a method, wherein the conjunctivodacryocystorhinostomy tube (200) includes borosilicate glass, steel, aluminum, plastic, or a combination thereof.

In some embodiments, the present invention features a method, wherein the solid layer (110) includes thermoplastic polyurethane, polycarbonate, polyethylene terephthalate, or polyethylene terephthalate-co-glycolate, or other polar thermoplastics such as polyamides, polyarylamides, polyketones, polyarylesters, or copolymers thereof.

In some embodiments, the present invention features a method further including applying dilute NaOH solution to the chitosan layer (120). In some embodiments, the present invention features a method further including rolling the antimicrobial structure (100) into a hollow cylindrical shape such that the plurality of nanopillars (125) face inwards in the hollow cylindrical shape and inserting the antimicrobial structure (100) into the conjunctivodacryocystorhinostomy tube (200). In some embodiments, the present invention features a method, wherein the conjunctivodacryocystorhinostomy tube (200) includes borosilicate glass, steel, aluminum, or plastic, In some embodiments, the present invention features a method, wherein the solid material includes thermoplastic polyurethane, polycarbonate, polyethylene terephthalate, or polyethylene terephthalate-co-glycolate, or other polar thermoplastics such as polyamides, polyarylamides, polyketones, polyarylesters, or copolymers thereof.

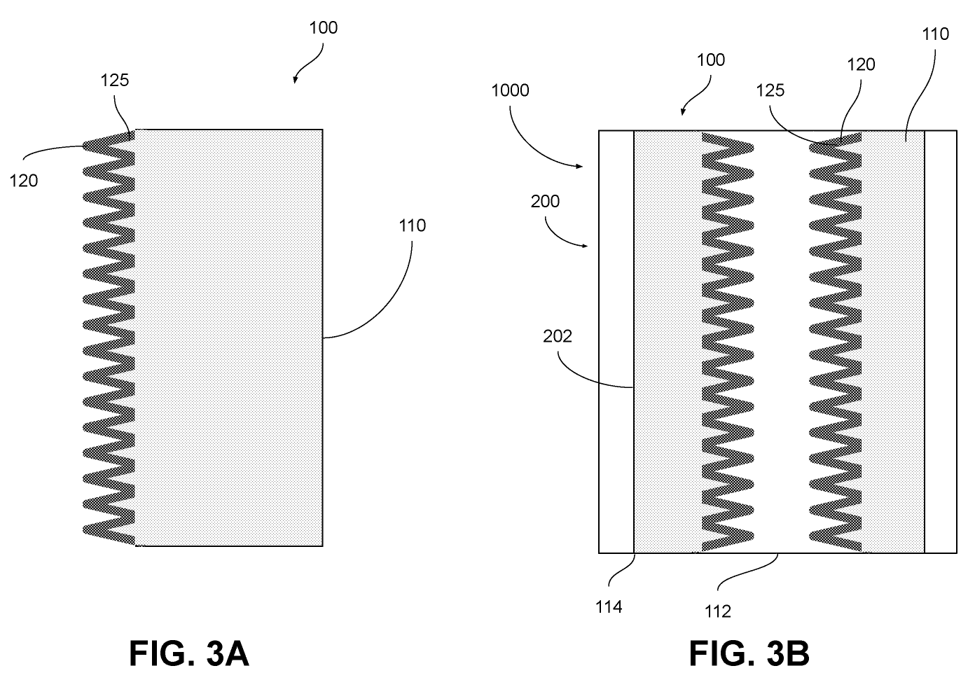

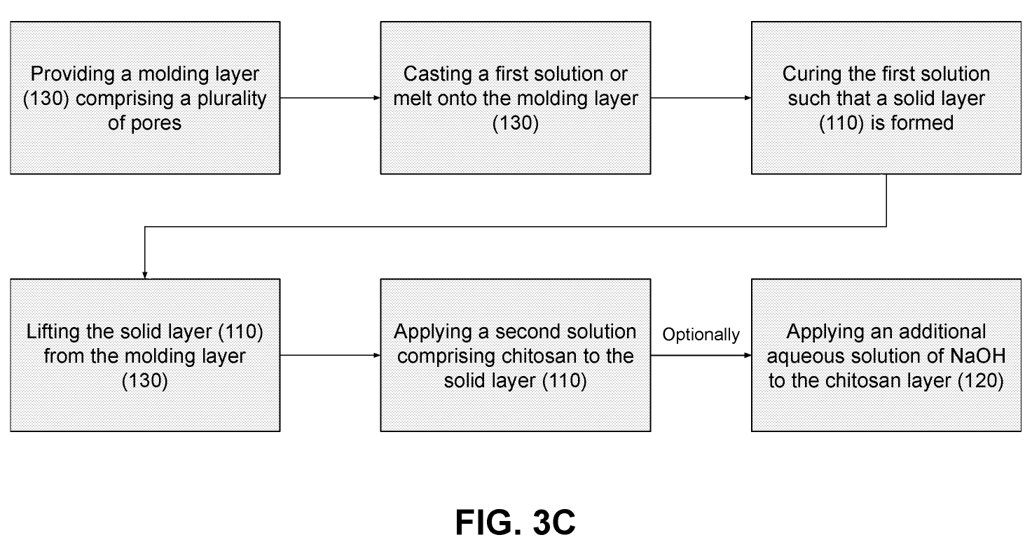

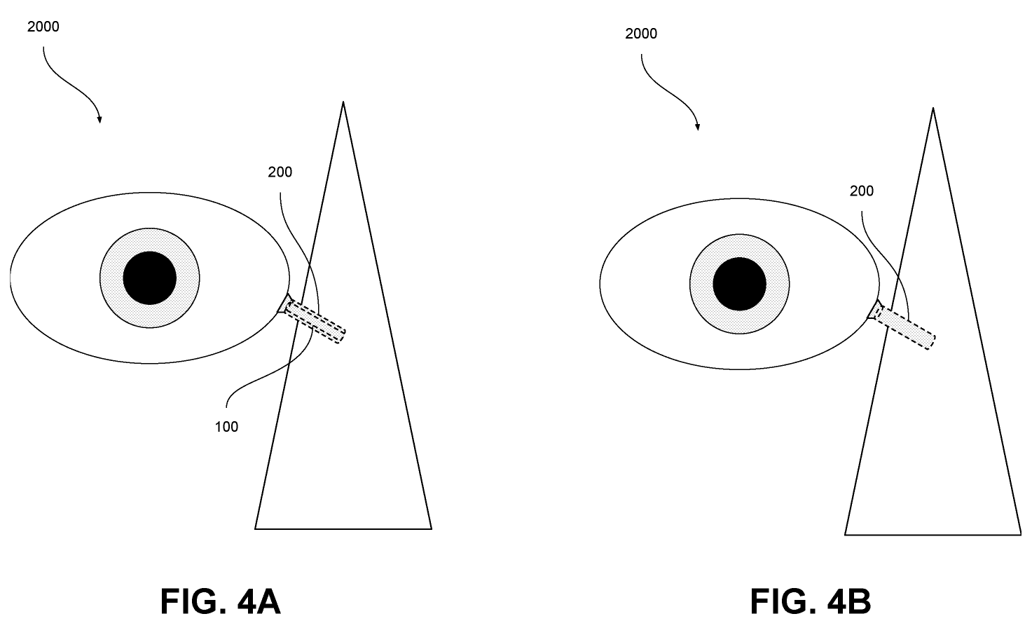

Referring now to FIG. 3A, the present invention features an antimicrobial structure (100) for insertion into a conjunctivodacryocystorhinostomy tube (200). In some embodiments, the structure (100) may comprise a first layer (110) comprising a solid material having a plurality of nanopillar protrusions. The structure (100) may further comprise a second layer (120) disposed on the first layer (110), comprising an antimicrobial chitosan material or another polar polymer disposed on the plurality of nanopillars.

In some embodiments, the solid material may comprise thermoplastic polyurethane, polycarbonate, polyethylene terephthalate, or polyethylene terephthalate-co-glycolate, or other polar thermoplastics such as polyamides, polyarylamides, polyketones, polyarylesters, or copolymers thereof. In some embodiments, the structure (100) may be configured to be rolled into a hollow cylindrical shape with the second layer (120) facing inwards and inserted into the conjunctivodacryocystorhinostomy tube (200). In some embodiments, the conjunctivodacryocystorhinostomy tube (200) may comprise borosilicate glass, steel, aluminum, plastic, a combination thereof, or any other durable material. In some embodiments, the plurality of nanopillars (125) may be formed on the first layer (120) comprising a plurality of V-shaped or cylindrical-shaped pores such that the plurality of nanopillars (125) are formed within the plurality of pores. In some embodiments, the molding layer (120) may comprise anodized aluminum oxide or anodized titanium oxide or a different polymer with V-shaped or cylindrical-shaped pores.

Referring now to FIG. 3B, the present invention features an antimicrobial system (1000) for draining tear buildup in a patient. In some embodiments, the system (1000) may comprise a conjunctivodacryocystorhinostomy tube (200). The system (1000) may further comprise an antimicrobial structure (100) rolled into a hollow cylindrical shape and disposed within the conjunctivodacryocystorhinostomy tube (200). The structure (100) may comprise a first layer (110) comprising a solid material having a plurality of nanopillar protrusions. The structure (100) may further comprise a second layer (120) disposed on the first layer (110), comprising an antimicrobial chitosan material or another polar polymer material disposed on a plurality of nanopillars (125).

In some embodiments, the solid material may comprise Thermoplastic Polyurethane (TPU). In some embodiments, the conjunctivodacryocystorhinostomy tube (200) may comprise borosilicate glass, steel, aluminum, plastic, a combination thereof, or any other durable material. In some embodiments, the plurality of nanopillars (125) may be formed on the solid layer (110) from a molding layer (130) comprising a plurality of V-shaped pores or cylindrical-shaped pores such that the plurality of nanopillars (125) are formed within the plurality of said pores. In some embodiments, the molding layer (130) may comprise anodized aluminum oxide or anodized titanium oxide

Referring now to FIG. 3C, the present invention features a method for fabricating an antimicrobial structure (100) for insertion into a conjunctivodacryocystorhinostomy tube (200). The method may comprise providing a molding layer (130) comprising a plurality of V-shaped pores or cylindrical-shaped pores. The method may further include casting a solution comprising the solution or melt of another polar polymer onto the molding layer (130) such that the solution or melt fills the plurality of V-shaped pores or cylindrical-shaped pores. The method may further comprise curing the solution, resulting in a solid layer (110) comprising a plurality of nanopillars. The method may further comprise lifting the solid layer (110) from the molding layer (130) and applying chitosan to the side of the solid layer (110) with nanopillars to result in the antimicrobial structure (100).

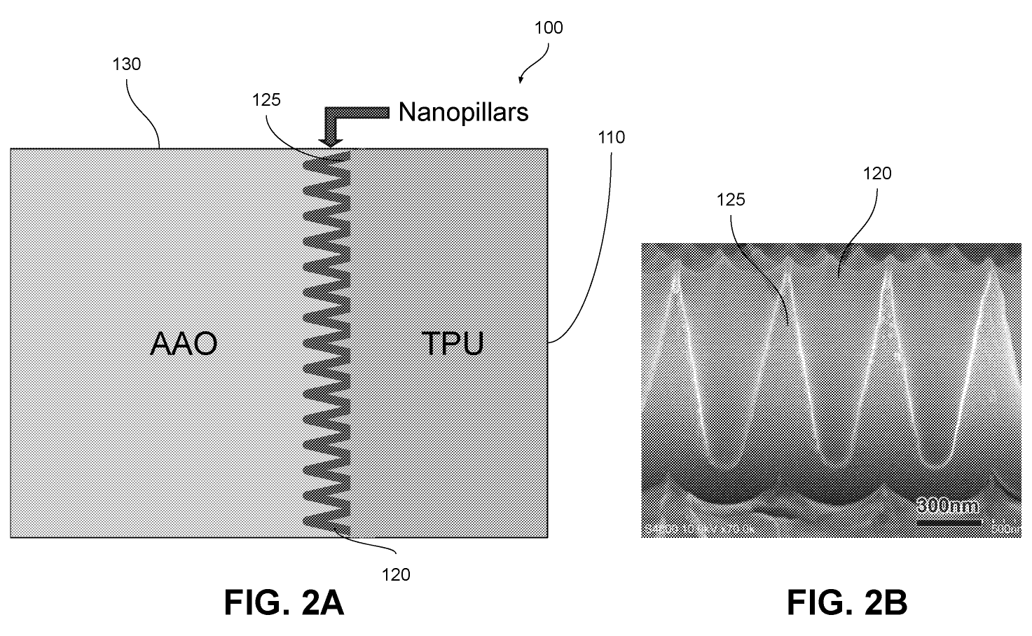

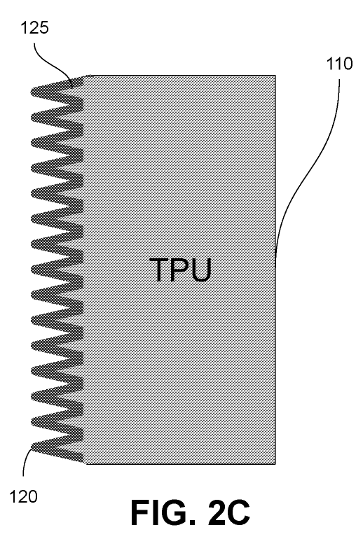

In some embodiments, the molding layer (130) may comprise anodized aluminum oxide or anodized titanium oxide. In some embodiments, the method may further comprise casting PFDTES onto the molding layer (130) and curing the PFDTES prior to applying the solution. In other embodiments, the antimicrobial coating and/or the solution from which this coating is derived is disposed directly on the inverted nano-pillars of the molding layer (130), as seen in FIG. 2C. In these embodiments, the substrate layer and/or the solution or melt from which this layer is derived is used to fill the inverted nanopillars with the antimicrobial coating, resulting in the antimicrobial structure.

In some embodiments, the solution may further comprise citric acid, acetic acid, hydrochloric acid, or a combination thereof. In some embodiments, the method may further comprise applying an additional aqueous solution of NaOH to the chitosan layer (120). In some embodiments, the method may further comprise rolling the antimicrobial structure (100) into a hollow cylindrical shape and inserting the antimicrobial structure (100) into the conjunctivodacryocystorhinostomy tube (200). In some embodiments, the conjunctivodacryocystorhinostomy tube (200) may comprise borosilicate glass, steel, aluminum, plastic, a combination thereof, or any other durable material.

In some embodiments, the antimicrobial structure (100) of the present invention may serve as an insert for a pre-set conjunctivodacryocystorhinostomy tube. In some embodiments, the antimicrobial structure (100) of the present invention may act as its own conjunctivodacryocystorhinostomy tube, self-standing in the soft tissue of the patient.

In some embodiments where the antimicrobial structure (100) acts as an insert, the substrate layer (110) may have an exterior surface configured to adhere to the interior of the conjunctivodacryocystorhinostomy tube. This adhesion may be achieved through adhesive material, a static attraction effect, or any other means for adhesion of a plastic material to another solid material (e.g., metal, plastic, glass).

In some embodiments where the antimicrobial structure (100) acts as a self-standing conjunctivodacryocystorhinostomy tube, the exterior of the substrate layer (110) may comprise properties suitable for residing long-term in the soft tissue of the patient. For example, the substrate layer (110) may be configured to have a degree of flexibility so as to withstand the flexure and compression of the eye muscles, the nasal muscles, or any adjacent muscles without displacing itself exterior to the body, behind soft tissue such that the first and/or second end(s) of the rube are blocked, into the nasal cavity, or in any other position where the structure no longer serves to drain tears from the eye to the nasal cavity. In some embodiments, this degree of flexibility may be achieved by implementing material and dimensions of the substrate layer with elasticity, the ability to store tension, and shape memory. This may allow the structure to accommodate the stress from the facial muscles without being crushed or blocking the fluid flow of tears entirely, springing back into shape when the structure is no longer being stressed. In some embodiments, the texture of the substrate layer may be smooth, rough, or a combination thereof (e.g., portions of smooth texture and portions of rough texture). In some embodiments, the structure may be shaped with one or more protrusions to maintain the structure in the soft tissue, such as a collar at the first and/or second end(s) of the tube shape, a helical protrusion, or a combination thereof.

In some embodiments, the antimicrobial structure (100) may be configured to maintain, at all times, an open, non-obstructed channel from the eyelids of the patient to the nasal cavity of the patient. This results in the lowest possible probability of blockage due to biofilm, allowing the structure to be maintained in the soft tissue for a longer period of time without the need for replacement. In some embodiments, the antimicrobial layer may comprise any material with the capability of rejecting the buildup of biofilm, either by repelling these contaminants, preventing the proliferation of bacteria and fungi, or a combination thereof.

### EXAMPLE

The following is a non-limiting example of the present invention. It is to be understood that said example is not intended to limit the present invention in any way. Equivalents or substitutes are within the scope of the present invention.

The chitosan (Ch) and Thermoplastic Polyurethane (TPU) (with hardness 72D) sandwich implemented in the present invention was fabricated through the following process. Anodized aluminum oxide (AAO) mold with V-shaped pores was cleaned in acetone, then EtOH (ethanol), dried in vacuum, and exposed to ultraviolet light (UV) to activate the surface. PFDTES in isooctane was cast onto AAO under a nitrogen atmosphere. Alternatively, a lecithin dispersion was applied to the AAO o. the thus-coated AAO mold was cured at 80 °C in vacuum. A 2% TPU polymer solution in dimethyl formamide (DMF) was cast onto the PFDTES- or lecithin coated AAO and dried at 80 °C in vacuum. The thus-coated sandwich was placed in UV for 10 min. An additional solution of 72D TPU was dropped onto the sandwich until the desired thickness was achieved. Alternatively, a solid film of the TPU could be adhered to the solvent cast film. In either process, pressure of 10 kPa was applied at the melting temperature of the given polymer, 215 °C in the case of 72D TPU, and the sandwich was allowed to cool. The sandwich was lifted from the AAO mold. A 2% chitosan solution in acetic acid was applied to the molded surface with the nanopillars and allowed to dry in the laboratory atmosphere. A 0.1 N NaOH was then applied to the chitosan side of the sandwich at room temperature. The sandwich was rinsed repeatedly and tested with pH paper until a pH of ~ 7 was reached.

Although there has been shown and described the preferred embodiment of the present invention, it will be readily apparent to those skilled in the art that modifications may be made thereto which do not exceed the scope of the appended claims. Therefore, the scope of the invention is limited only by the following claims. In some embodiments, the figures presented in this patent application are drawn to scale, including the angles, ratios of dimensions, etc. In some embodiments, the figures are representative only, and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. An antimicrobial structure (100) configured to create a channel in soft tissue from an eyelid to a nasal passage of a patient (2000) for tear liquid drainage, the antimicrobial structure (100) comprising:
a) a substrate layer (110) comprising a solid material arranged in a hollow cylindrical shape having an interior surface (112) and an exterior surface (114), and a plurality of nanopillars (125); and
b) an antimicrobial layer (120) disposed on the interior surface of the substrate layer (110), comprising an antimicrobial film disposed on the plurality of nanopillars (125).

2. The antimicrobial structure (100) of claim 1, wherein the solid material comprises thermoplastic polyurethane, polycarbonate, polyethylene terephthalate, polyethylene terephthalate-co-glycolate, polyamides, polyarylamides, polyketones, polyarylesters, or any copolymers thereof.

3. The antimicrobial structure (100) of claim 1, wherein the antimicrobial structure (100) is configured to be inserted into a conjunctivodacryocystorhinostomy tube (200) having an interior surface (202) such that the exterior surface (114) of the substrate layer (110) adheres to the interior surface (202) of the conjunctivodacryocystorhinostomy tube (200).

4. The antimicrobial structure (100) of claim 1, wherein the antimicrobial structure (100) is configured to be inserted directly into the soft tissue and lacrimal bone of the patient (2000) such that the exterior surface (114) of the substrate layer (110) contacts the soft tissue and the lacrimal bone of the patient (2000).

5. The antimicrobial structure (100) of claim 1, wherein the plurality of nanopillars (125) are formed on the solid layer (110) from a molding layer (130) comprising a plurality of pores comprising V-shaped pores, cylindrical-shaped pores, or a combination thereof such that the plurality of nanopillars (125) are formed within the plurality of pores.

6. The antimicrobial structure (100) of claim 5, wherein the molding layer (130) comprises anodized aluminum oxide, or anodized titanium oxide.

7. An antimicrobial system (1000) configured to create a channel in soft tissue from an eyelid to a nasal passage of a patient (2000) for tear liquid drainage, the antimicrobial system (1000) comprising:
a) a conjunctivodacryocystorhinostomy tube (200); and
b) an antimicrobial structure (100) rolled into a hollow cylindrical shape and disposed within the conjunctivodacryocystorhinostomy tube (200), the antimicrobial structure (100) comprising:
i) a substrate layer (110) comprising a solid material arranged in a hollow cylindrical shape having an interior surface (112) and an exterior surface (114), and a plurality of nanopillars (125); and
ii) an antimicrobial layer (120) disposed on the interior surface of the substrate layer (110), comprising an antimicrobial film disposed on the plurality of nanopillars (125).

8. The antimicrobial system (1000) of claim 7, wherein the solid material comprises thermoplastic polyurethane, polycarbonate, polyethylene terephthalate, polyethylene terephthalate-co-glycolate, polyamides, polyarylamides, polyketones, polyarylesters, or any copolymers thereof.

9. The antimicrobial system (1000) of claim 7, wherein the conjunctivodacryocystorhinostomy tube (200) comprises borosilicate glass, steel, aluminum, plastic, or a combination thereof.

10. The antimicrobial system (1000) of claim 7, wherein the plurality of nanopillars (125) are formed on the solid layer (110) from a molding layer (130) comprising a plurality of pores comprising V-shaped pores, cylindrical-shaped pores, or a combination thereof such that the plurality of nanopillars (125) are formed within the plurality of pores.

11. The antimicrobial system (1000) of claim 10, wherein the molding layer (130) comprises anodized aluminum oxide, anodized titanium oxide, or a combination thereof.

12. A method for fabricating an antimicrobial structure (100) for insertion into a conjunctivodacryocystorhinostomy tube (200), the method comprising:
a) providing a molding layer (130) comprising a plurality of pores;
b) casting a first solution or melt onto the molding layer (130);
c) curing the first solution or melt such that a solid layer (110) is formed, the solid layer (110) comprising a plurality of nanopillars (125) formed in the plurality of pores;
d) lifting the solid layer (110) from the molding layer (130) to result in the antimicrobial structure (100); and
e) applying a second solution comprising chitosan to the solid layer (110), resulting in the antimicrobial structure (100).

13. The method of claim 12, wherein the molding layer (130) comprises anodized aluminum oxide, or anodized titanium oxide.

14. The method of claim 12, wherein the first solution or melt comprises an emulsion of lecithin, or H,1H,2H,2H-Perfluorodecyltriethoxysilane (PFDTES), or a combination thereof.

15. The method of claim 12, wherein the second solution further comprises citric acid, acetic acid, or a combination thereof.

16. The method of claim 12 further comprises applying pressure to the antimicrobial structure (100).

17. The method of claim 12 further comprising applying a dilute NaOH solution to the chitosan layer (120), applying oxygen plasma treatment to the chitosan layer (120), or a combination thereof.

18. The method of claim 12 further comprising rolling the antimicrobial structure (100) into a hollow cylindrical shape such that the plurality of nanopillars (125) face inwards in the hollow cylindrical shape, and inserting the antimicrobial structure (100) into the conjunctivodacryocystorhinostomy tube (200).

19. The method of claim 18, wherein the conjunctivodacryocystorhinostomy tube (200) comprises borosilicate glass, steel, aluminum, plastic, or a combination thereof.

20. The method of claim 12, wherein the solid layer (110) comprises thermoplastic polyurethane, polycarbonate, polyethylene terephthalate, polyethylene terephthalate-co-glycolate, polyamides, polyarylamides, polyketones, polyarylesters, or any copolymers thereof.
